(19) United States
(12) Patent Application Publication
(10) Pub. No.: US 2020/0011578 A1
(43) Pub. Date: Jan. 9, 2020

(54) REFRIGERANT CIRCULATION DEVICE AND REFRIGERANT CIRCULATION METHOD

(71) Applicant: MITSUBISHI HEAVY INDUSTRIES THERMAL SYSTEMS, LTD., Tokyo (JP)

(72) Inventors: Ryosuke SUEMITSU, Tokyo (JP); Kenji UEDA, Tokyo (JP); Kazuki WAJIMA, Tokyo (JP); Naoki KOBAYASHI, Tokyo (JP)

(73) Assignee: MITSUBISHI HEAVY INDUSTRIES THERMAL SYSTEMS, LTD., Tokyo (JP)

(21) Appl. No.: 16/493,176

(22) PCT Filed: Mar. 8, 2018
(86) PCT No.: PCT/JP2018/009072

§ 371 Date: Sep. 11, 2019

(30) Foreign Application Priority Data
Mar. 29, 2017 (JP) 2017-064886

(51) Int. Cl. F25B 43/00; F25B 41/00; F25B 41/06; F25B 30/02; C09K 5/04; C07C 21/18

(52) U.S. Cl. CPC F25B 43/00; F25B 41/003; F25B 41/062; F25B 30/02; C09K 2205/126; C07C 21/18; F25B 2300/00; C09K 2205/122; C09K 2205/40; C09K 5/044

(57) ABSTRACT

The objective of the present invention is to provide a refrigerant circulation device and a refrigerant circulation method with which a change in pressure inside the device can be avoided and performance stability and stable operation are achieved even when a HFO or a HCFO refrigerant is used. In the refrigerant circulation device (1), a refrigerant circulation circuit for circulating a refrigerant is formed by connecting a compressor (3), a condenser (5), an expansion valve (7), and an evaporator (9) through primary pipes (11a, 11b, 11c, 11d), and the refrigerant circulation circuit is charged with a refrigerant containing hydrofluoroolefin or hydrochlorofluoroolefin having a carbon-carbon double bond in the molecular structure. The refrigerant circulation device (1) is provided with a recovery catalyst that is arranged so as to be capable of contacting the refrigerant in the refrigerant circulation circuit, and is for returning, to the pre-isomerization state, an isomer formed by isomerization of the hydrofluoroolefin or hydrochlorofluoroolefin contained in the initial refrigerant with which the refrigerant circulation circuit is initially charged.

### TECHNICAL FIELD

The present invention relates to a refrigerant circulation device and a refrigerant circulation method.

### BACKGROUND ART

In the related art, in a turbo heat pump, a hydrofluorocarbon (HFC) refrigerant is used. However, the global warming potential (GWP) of the HFC refrigerant is extremely high at several hundreds to thousands.

The use of a refrigerant having a high GWP is not desirable from the viewpoint of global environment protection. Therefore, transition to a refrigerant having a low GWP is required.

As the refrigerant having a low GWP, a hydrofluoroolefin (HFO) or hydrochlorofluoroolefin (HCFO) refrigerant is known (refer to PTLs 1 and 2). HFO and HCFO have a carbon-carbon double bond in the molecular structure. HFO and HCFO may have a stereoisomer (cis-trans isomer).

### CITATION LIST

### Patent Literature

[PTL 1] Japanese Unexamined Patent Application Publication No. 2015-083899

[PTL 2] Japanese Unexamined Patent Application Publication No. 2013-107848

### SUMMARY OF INVENTION

### Technical Problem

A HFO or HCFO refrigerant has lower stability than HFC and is isomerized when exposed to a high-temperature environment. The cis-trans isomer has the same composition but has different physical properties such as boiling point, heat transfer, or flow characteristics. Therefore, when isomerization progresses, heat transfer characteristics and flow characteristics of the refrigerant change and the pressure of the refrigerant changes. For example, when an isomer (low-pressure stereoisomer) having a high boiling point is isomerized such the amount of an isomer having a low boiling point (high-pressure stereoisomer) in the refrigerant increases, the saturation pressure of the refrigerant increases.

A device such as a heat pump device is designed to endure the saturation pressure of a refrigerant that is initially charged as a saturation pressure. As the refrigerant becomes isomerized with the passage of the operation time of the heat pump, the pressure inside the device increases, which leads to damages to the device.

Under circumstances where physical properties of a refrigerant at the time of charging change during operation, a stable heat cycle cannot be maintained.

The present invention has been made in consideration of the above-described circumstances, and an object thereof is to provide a refrigerant circulation device and a refrigerant circulation method with which a change in pressure inside the device can be avoided, the performance of a refrigerant can be stabilized, and stable operation can be achieved even when a HFO or a HCFO refrigerant is used.

### Solution to Problem

In order to achieve the object, the refrigerant circulation device and the refrigerant circulation method according to the present invention adopts the following means.

According to an aspect of the present invention, there is provided a refrigerant circulation device in which a refrigerant circulation circuit for circulating a refrigerant is formed by connecting a compressor, a condenser, an expansion valve, and an evaporator through primary pipes, and the refrigerant circulation circuit is charged with a refrigerant containing hydrofluoroolefin (HFO) or hydrochlorofluoroolefin (HCFO) having a carbon-carbon double bond in a molecular structure, the refrigerant circulation device including: a recovery catalyst that is arranged so as to be capable of contacting the refrigerant in the refrigerant circulation circuit and returns, to a pre-isomerization state, an isomer formed by isomerization of hydrofluoroolefin or hydrochlorofluoroolefin contained in the initial refrigerant with which the refrigerant circulation circuit is initially charged.

The refrigerant that circulates in the refrigerant circulation circuit contacts the recovery catalyst during circulation. Even when hydrofluoroolefin or hydrochlorofluoroolefin contained in the initial refrigerant with which the refrigerant circulation circuit is initially charged is isomerized in the refrigerant circulation circuit during circulation, hydrofluoroolefin or hydrochlorofluoroolefin returns to the pre-isomerization state by contacting the recovery catalyst. As a result, a change in thermal physical properties of the refrigerant can be suppressed, and a stable heat cycle can be maintained.

In the aspect of the present invention, the recovery catalyst may be arranged between the compressor and the condenser.

In the aspect of the present invention, the recovery catalyst may be arranged between the condenser and the evaporator.

In the aspect of the present invention, the refrigerant circulation device may include: a driving machine that drives the compressor through an accelerator; an introduction passage that connects the condenser and the driving machine to each other and guides the condensed refrigerant to the driving machine; and a return passage that connects a primary pipe between the expansion valve and the evaporator to the driving machine and returns the refrigerant having passed through the driving machine to the primary pipe, in which the recovery catalyst is arranged in the return passage or around the driving machine.

A region between the compressor and the condenser, a region between the condenser and the evaporator, and a region in the return passage or around the driving machine are regions (high-temperature regions) where the operation temperature of the refrigerant is high. Here, the high temperature refers to 150° C. or higher. In the high-temperature region, the refrigerant is likely to be isomerized. By providing the recovery catalyst in the high-temperature region, an isomer of which the amount increases by isomerization returns to the pre-isomerization state, and a change in thermal physical properties of the refrigerant can be suppressed.

According to another aspect of the present invention, there is provided a refrigerant circulation method for a refrigerant circulation device in which a refrigerant circulation circuit for circulating a refrigerant is formed by connecting a compressor, a condenser, an expansion valve, and an evaporator through primary pipes, and the refrigerant circulation circuit is charged with a refrigerant containing hydrofluoroolefin or hydrochlorofluoroolefin having a carbon-carbon double bond in a molecular structure, the refrigerant circulation method including: arranging a recovery catalyst to a region of the refrigerant circulation circuit where an operation temperature of the refrigerant is 150° C. or higher, the recovery catalyst returning, to a pre-isomerization state, an isomer formed by isomerization of hydrofluoroolefin or hydrochlorofluoroolefin contained in the initial refrigerant with which the refrigerant circulation circuit is initially charged; and bringing the refrigerant that circulates in the refrigerant circulation circuit into contact with the recovery catalyst.

In the aspect of the present invention, the region where the operation temperature of the refrigerant is 150° C. or higher may be present between the compressor and the condenser.

In the aspect of the present invention, the region where the operation temperature of the refrigerant is 150° C. or higher may be present between the condenser and the evaporator.

In the aspect of the present invention, the refrigerant circulation device may include: a driving machine that drives the compressor through an accelerator; an introduction passage that connects the condenser and the driving machine to each other and guides the condensed refrigerant to the driving machine; and a return passage that connects a primary pipe between the expansion valve and the evaporator to the driving machine and returns the refrigerant having passed through the driving machine to the primary pipe, in which the region where the operation temperature of the refrigerant is 150° C. or higher is present in the return passage or around the driving machine.

### Advantageous Effects of Invention

With the refrigerant circulation device and the refrigerant circulation method according to the present invention, a change in pressure inside the device can be avoided, the performance of a refrigerant can be stabilized, and stable operation can be achieved even when a HFO or a HCFO refrigerant is used.

### BRIEF DESCRIPTION OF DRAWINGS

FIG. 1 is a schematic configuration diagram illustrating an example of a heat pump device according to one embodiment.

### DESCRIPTION OF EMBODIMENTS

In a heat pump device according to an embodiment, a refrigerant circulation circuit is charged with a refrigerant (hereinafter, referred to as “HFO refrigerant” or “HCFO refrigerant”) containing hydrofluoroolefin (HFO) or hydrochlorofluoroolefin (HCFO). HFO or HCFO is a refrigerant having a carbon-carbon double bond in the molecular structure.

It is preferable that the refrigerant contains HFO or HCFO as a major component. The content of HFO or HCFO in the refrigerant is higher than 50 mass %, preferably 75 mass % or higher, and still more preferably 90 mass % or higher.

Specifically, hydrofluoroolefin (HFO) is (Z)-1,3,3,3-tetrafluoro-1-propene (HFO1234ze(Z)), (Z)-1,1,1,4,4,4-hexafluoro-2-butene (HFO1336mzz(Z)), (E)-1,1,1,4,4,5,5,5-octafluoropent-2-ene (HFO1438mzz(E)), or (Z)-1,1,1,4,4,5,5,5-octafluoropent-2-ene (HFO-1438mzz(Z)).

Specifically, hydrochlorofluoroolefin (HCFO) is (E)-1-chloro-3,3,3-trifluoropropene (HCFO1233zd(E)), (Z)-1-chloro-3,3,3-trifluoropropene (HCFO1233zd(Z)), or (Z)-1,2-dichloro-3,3,3-trifluoropropene (HCFO1223xd(Z)).

The purity of HFO or HCFO is preferably 97 mass % or higher, more preferably 99 mass % or higher, and still more preferably 99.9 mass % or higher.

The refrigerant may contain additives. Examples of the additives include halocarbons, other hydrofluorocarbons (HFC), alcohols, and saturated hydrocarbons.

<Halocarbons and Other Hydrofluorocarbons>

Examples of the halocarbons include methylene chloride, trichloroethylene, and tetrachloroethylene that contain a halogen atom. Examples of the hydrofluorocarbons include difluoromethane (HFC-32), 1,1,1,2,2-pentafluoroethane (HFC-125), fluoroethane (HFC-161), 1,1,2,2-tetrafluoroethane (HFC-134), 1,1,1,2-tetrafluoroethane (HFC-134a), 1,1,1-trifluoroethane (HFC-143a), difluoroethane (HFC-152a), 1,1,1,2,3,3,3-heptafluoropropane (HFC-227ea), 1,1,1,2,3-heptafluoropropane (HFC-236ea), 1,1,1,3,3,3-hexafluoropropane (HFC-236fa), 1,1,1,3,3-pentafluoropropane (HFC-245fa), 1,1,1,2,3-pentafluoropropane (HFC-245eb), 1,1,2,2,3-pentafluoropropane (HFC-245ca), 1,1,1,3,3-pentafluorobutane (HFC-365mfc), 1,1,1,3,3,3-hexafluoroisobutane (HFC-356mmz), and 1,1,1,2,2,3,4,5,5,5-decafluoropentane (HFC-43-10-mee).

<Alcohols>

Examples of the alcohols include alcohols having 1 to 4 carbon atoms such as methanol, ethanol, n-propanol, i-propanol, n-butanol, i-butanol, 2,2,2-trifluoroethanol, pentafluoropropanol, tetrafluoropropanol, and 1,1,1,3,3,3-hexafluoro-2-propanol.

<Saturated Hydrocarbons>

Examples of the saturated hydrocarbon include a saturated hydrocarbon having 3 to 8 carbon atoms such as a mixture of one or more compounds selected from the group consisting of propane, n-butane, i-butane, neopentane, n-pentane, i-pentane, cyclopentane, methylcyclopentane, n-hexane, and cyclohexane. Among these, neopentane, n-pentane, i-pentane, cyclopentane, methylcyclopentane, n-hexane, or cyclohexane is preferable.

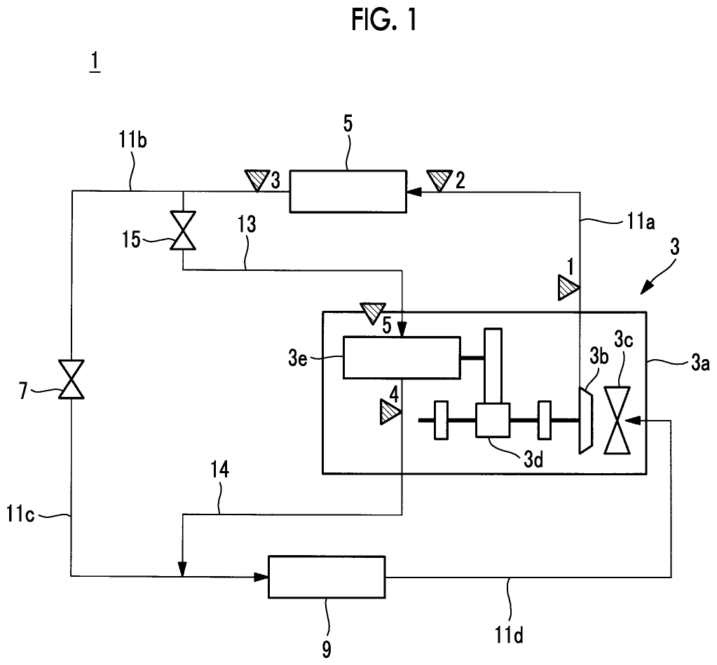

FIG. 1 is a schematic configuration diagram illustrating an example of a heat pump device (refrigerant circulation device) that is charged with the refrigerant.

The heat pump device 1 includes: a compressor 3 that compresses the refrigerant; a condenser 5 that condenses the refrigerant compressed by the compressor 3; an expansion valve 7 that expands the liquid refrigerant from the condenser 5; an evaporator 9 that evaporates the refrigerant expanded by the expansion valve 7; and a recovery catalyst that returns the refrigerant containing isomerized HFO or HCFO into the initial state.

The compressor 3, the condenser 5, the expansion valve 7, and the evaporator 9 are connected through primary pipes (11a, 11b, 11c, and 11d) to form a closed system (heat pump cycle/refrigerant circulation circuit) for circulating the refrigerant. Each of the members constituting the heat pump device 1 is designed to endure a pressure from the refrigerant. The heat pump device 1 can output warm water at 200° C.

The compressor 3 is a centrifugal compressor capable of obtaining a high pressure ratio. The compressor 3 can increase the temperature of the refrigerant up to about 230° C. The compressor 3 includes: an impeller 3b that is provided inside a casing 3a; an inlet vane 3c that adjusts the flow rate of the refrigerant to be suctioned; an accelerator 3d, and a driving machine 3e.

The impeller 3b is rotated by the driving machine 3e through the accelerator 3d. The driving machine 3e is an electric motor. The electric motor may also operate such that the rotational frequency is variable by an inverter device. The rotational frequency of the driving machine 3e is controlled by a control unit (not illustrated).

One end of the primary pipe 11a is connected to an outlet side of the compressor 3. Another end of the primary pipe 11a is connected to an inlet side of the condenser 5.

The condenser 5 has a structure in which latent heat of condensation of the refrigerant is captured by cooling water. As the condenser 5, a shell and tube heat exchanger is suitably used, but a blade heat exchanger may also be used. The liquid refrigerant condensed by the condenser 5 passes through the primary pipe 11b and is guided to the expansion valve 7.

One end of the primary pipe 11b is connected to an outlet side of the condenser 5. Another end of the primary pipe 11b is connected to the expansion valve 7.

The expansion valve 7 is an electronic expansion valve or an electric ball valve, and the opening degree thereof is controlled by a control unit (not illustrated). The liquid refrigerant flowing through the primary pipe is decompressed and expanded by the expansion valve 7.

One end of the primary pipe 11c is connected to the expansion valve 7. Another end of the primary pipe 11c is connected to an inlet side of the evaporator 9.

The evaporator 9 includes a heat transfer pipe (not illustrated) inside a container. A chilled water pipe (not illustrated) is connected to the heat transfer pipe such that chilled water can be supplied to an external thermal load. The chilled water is cooled by latent heat of evaporation of the liquid refrigerant inside the evaporator 9 when flowing through the heat transfer pipe. The evaporator 9 is, for example, a shell and tube heat exchanger.

One end of the primary pipe 11d is connected to an outlet side of the evaporator 9. Another end of the primary pipe 11d is connected to the inlet vane 3c of the compressor 3.

A control unit (not illustrated) of the heat pump device 1 is provided on a control board inside a control panel of the heat pump device 1 and includes a CPU and a memory. The control unit calculates each of control amounts by digital operation every control period based on a cooling water temperature, a refrigerant pressure, a chilled water inlet-outlet temperature, and the like.

The heat pump device 1 includes: an introduction passage 13 that guides a part of the liquid refrigerant condensed by the condenser 5 from the primary pipe 11b to the driving machine 3e; and a return passage 14 that returns the refrigerant having passed through the driving machine 3e to the primary pipe 11c. “Having passed through the driving machine 3e” refers to the refrigerant having flowed after the contact with the driving machine 3e or the refrigerant having passed through the periphery of the driving machine 3e (a range of the casing 3a).

One end of the introduction passage 13 is connected to the primary pipe 11b on the outlet side of the condenser 5, and another end of the introduction passage 13 is connected to the driving machine 3e. The other end of the introduction passage 13 is not necessarily directly connected to the driving machine 3e, and may connect the primary pipe 11b and the driving machine 3e to each other in a state where the driving machine 3e can be cooled using the condensed liquid refrigerant. For example, the introduction passage 13 may be connected to the casing 3a positioned near the driving machine 3e.

A throttle valve 15 is provided halfway the introduction passage 13. The opening degree of the throttle valve 15 is controlled by a control unit (not illustrated) such that the driving machine 3e is appropriately cooled.

One end of the return passage 14 is connected to the driving machine 3e, and another end of the return passage 14 is connected to the primary pipe 11c on the inlet side of the evaporator 9. The one end of the return passage 14 is not necessarily directly connected to the driving machine 3e and may connect the driving machine 3e and the primary pipe 11c to each other in a state where the refrigerant having passed through the driving machine 3e can return to the primary pipe 11c. For example, the return passage 14 may be connected to the casing 3a present at a position facing the other end of the introduction passage 13 with the driving machine 3e interposed therebetween.

The recovery catalyst is arranged inside the refrigerant circulation circuit so as to be capable of contacting the refrigerant. ∇₁ to ∇₅ of FIG. 1 illustrate positions suitable for the arrangement of the recovery catalyst.

The recovery catalyst is arranged in a range where the operation temperature of the refrigerant is 150° C. or higher, preferably 175° C. or higher, and still more preferably 200° C. or higher. The recovery catalyst can be arranged at one region or a plurality of regions inside the refrigerant circulation circuit.

Examples of the region where the operation temperature of the refrigerant is 150° C. or higher include a region between the compressor 3 and the condenser 5, a region between the condenser 5 and the expansion valve 7, and a region in the return passage (∇₄) or around the driving machine (∇₅). The region between the compressor 3 and the condenser 5 includes a compressor outlet (∇₁), a condenser inlet (∇₂), and the primary pipe 11a. The region between the condenser 5 and the expansion valve 7 include a condenser outlet (∇₃) and the primary pipe 11b. The region around the driving machine includes an outer periphery and an end surface of the driving machine 3e and a range of the casing (in particular, near the driving machine 3e) that accommodates the driving machine 3e.

Examples of the region where the operation temperature of the refrigerant is 175° C. or higher include a region between the condenser 5 and the expansion valve 7 and a region in the return passage (∇₄) or around the driving machine (∇₅).

A region where the operation temperature of the refrigerant is 200° C. or higher is present between the condenser 5 and the expansion valve 7.

The recovery catalyst has properties capable of returning, to a pre-isomerization state, an isomer formed by isomerization of hydrofluoroolefin or hydrochlorofluoroolefin contained in a refrigerant (initial refrigerant) with which the refrigerant circulation circuit is initially charged.

The recovery catalyst can be appropriately selected according to the kind of the initial refrigerant. The recovery catalyst is, for example, a metal fluoride, a metal oxide, or a fluorinated metal oxide.

The metal fluoride is, for example, aluminum fluoride, chromium fluoride, titanium fluoride, manganese fluoride, iron fluoride, nickel fluoride, cobalt fluoride, magnesium fluoride, zirconium fluoride, or antimony fluoride.

The metal oxide is, for example, an oxide containing one kind of metal or two or more kinds of metal of which 50% or higher of metal atoms are composed of aluminum. Examples of the metal other than aluminum include chromium, titanium, manganese, iron, nickel, cobalt, magnesium, zirconium, and antimony. More specifically, the metal oxide may be a composite oxide of alumina and chromium, a composite oxide of alumina and zirconia, a composite oxide of alumina and titania, or a composite oxide of alumina and magnesia.

The fluorinated metal oxide is a metal oxide that is fluorinated by being brought into contact with a fluorinating agent. Examples of the fluorinated metal oxide include fluorinated alumina, fluorinated titanium oxide, fluorinated manganese oxide, fluorinated iron oxide, fluorinated nickel oxide, fluorinated cobalt oxide, fluorinated magnesia, fluorinated zirconia, fluorinated antimony oxide, and fluorinated chromia.

The recovery catalyst may have a shape that increases the contact area with the refrigerant. For example, the recovery catalyst has a shape such as a porous plate or a wire mesh. In a region where the liquid refrigerant is present, the recovery catalyst having a porous plate shape may be arranged. In a region where the gaseous refrigerant is present, the recovery catalyst having a wire mesh shape may be arranged. The recovery catalyst may be arranged toward a direction intersecting a refrigerant flowing direction such that the refrigerant passes through pores (or meshes) of the recovery catalyst.

In the heat pump device 1, the compressor may be a two-stage compressor including two impellers.

For example, the heat pump device 1 may include: a lubricant circulation unit (not illustrated) that circulates a lubricant to the casing 3a accommodating the accelerator 3d; an air bleeding device (not illustrated) that extracts air from the refrigerant circulation circuit; and a refrigerant supply pipe (not illustrated) for supplying the refrigerant to the refrigerant circulation circuit.

Next, the operation and effects of the heat pump device having the above-described configuration will be described. The low-pressure gaseous refrigerant sucked from the evaporator 9 is compressed by the impeller 3b of the compressor 3 and is converted into the high-pressure gaseous refrigerant.

The high-pressure gaseous refrigerant discharged from the compressor 3 is guided to the condenser 5 through the primary pipe 11a. In the condenser 5, the high-pressure gaseous refrigerant is cooled substantially at the same pressure and is converted into the high-pressure liquid refrigerant. Most of the high-pressure liquid refrigerant is guided to the expansion valve 7 through the primary pipe 11b, and a part of the high-pressure liquid refrigerant is guided to the driving machine 3e through the introduction passage 13.

The high-pressure liquid refrigerant guided to the expansion valve 7 is isenthalpically expanded up to a low pressure and then is guided to the evaporator 9 through the primary pipe 11c.

The liquid refrigerant guided to the evaporator 9 is evaporated due to heat exchange with the chilled water flowing through the heat transfer pipe and is converted into the low-pressure gaseous refrigerant. The low-pressure gaseous refrigerant flows to the inlet vane 3c of the compressor 3 through the primary pipe 11d and is compressed again by the impeller 3b.

The high-pressure liquid refrigerant guided to the introduction passage 13 cools the driving machine 3e and then is guided to the return passage 14. The opening degree of the throttle valve 15 is regulated by a control unit (not illustrated) such that the refrigerant has a desired temperature. By providing the throttle valve 15, even in the heat pump device 1 in which a region where the temperature of the refrigerant is at a high temperature of about 200° C. is present, the driving machine 3e can be cooled by the liquid refrigerant condensed by the condenser 5.

The refrigerant with which the refrigerant circulation circuit is charged contacts the recovery catalyst in the process of circulation. When the refrigerant contains an isomer formed by isomerization of hydrofluoroolefin or hydrochlorofluoroolefin contained in the initial refrigerant, the refrigerant returns to the pre-isomerization state by contacting the recovery catalyst. As a result, the isomer that is not dominantly present in the initial refrigerant is inhibited from being predominant in the heat pump, a change in pressure inside the device can be avoided, the performance of the refrigerant can be stabilized, and the heat pump device 1 can be stably operated.

For example, it is assumed that a refrigerant containing HCFO1233zd(E) as a major component is charged as the initial refrigerant and fluorinated alumina is arranged as the recovery catalyst. HCFO1233zd(E) contained in the refrigerant may be isomerized into HCFO1233zd(Z) by being exposed to a high temperature of 150° C. or higher in the refrigerant circulation circuit. In the heat pump device 1 according to the embodiment, the recovery catalyst is arranged in the region where the operation temperature of the refrigerant is 150° C. or higher, and when the refrigerant containing HCFO1233zd(Z) as an isomer contacts the recovery catalyst, HCFO1233zd(Z) returns to HCFO1233zd(E).

### REFERENCE SIGNS LIST

1: heat pump device (refrigerant circulation device)
3: compressor
3a: casing
3b: impeller
3c: inlet vane
3d: accelerator
3e: driving machine
5: condenser
7: expansion valve
9: evaporator
11a, 11b, 11c, 11d: primary pipe
13: introduction passage
14: return passage
15; throttle valve

1. A refrigerant circulation device in which a refrigerant circulation circuit for circulating a refrigerant is formed by connecting a compressor, a condenser, an expansion valve, and an evaporator through primary pipes, and the refrigerant circulation circuit is charged with a refrigerant containing hydrofluoroolefin or hydrochlorofluoroolefin having a carbon-carbon double bond in a molecular structure, the refrigerant circulation device comprising:
a recovery catalyst that is arranged so as to be capable of contacting the refrigerant in the refrigerant circulation circuit and returns, to a pre-isomerization state, an isomer formed by isomerization of hydrofluoroolefin or hydrochlorofluoroolefin contained in the initial refrigerant with which the refrigerant circulation circuit is initially charged.

2. The refrigerant circulation device according to claim 1,
wherein the recovery catalyst is arranged between the compressor and the condenser.

3. The refrigerant circulation device according to claim 1,
wherein the recovery catalyst is arranged between the condenser and the evaporator.

4. The refrigerant circulation device according to claim 1, further comprising:
a driving machine that drives the compressor through an accelerator;
an introduction passage that connects the condenser and the driving machine to each other and guides the condensed refrigerant to the driving machine; and
a return passage that connects a primary pipe between the expansion valve and the evaporator to the driving machine and returns the refrigerant having passed through the driving machine to the primary pipe,
wherein the recovery catalyst is arranged in the return passage or around the driving machine.

5. A refrigerant circulation method for a refrigerant circulation device in which a refrigerant circulation circuit for circulating a refrigerant is formed by connecting a compressor, a condenser, an expansion valve, and an evaporator through primary pipes, and the refrigerant circulation circuit is charged with a refrigerant containing hydrofluoroolefin or hydrochlorofluoroolefin having a carbon-carbon double bond in a molecular structure, the refrigerant circulation method comprising:
arranging a recovery catalyst to a region of the refrigerant circulation circuit where an operation temperature of the refrigerant is 150° C. or higher, the recovery catalyst returning, to a pre-isomerization state, an isomer formed by isomerization of hydrofluoroolefin or hydrochlorofluoroolefin contained in the initial refrigerant with which the refrigerant circulation circuit is initially charged; and
bringing the refrigerant that circulates in the refrigerant circulation circuit into contact with the recovery catalyst.

6. The refrigerant circulation method according to claim 5,
where the region where the operation temperature of the refrigerant is 150° C. or higher is present between the compressor and the condenser.

7. The refrigerant circulation method according to claim 5,
where the region where the operation temperature of the refrigerant is 150° C. or higher is present between the condenser and the evaporator.

8. The refrigerant circulation method according to claim 5,
wherein the refrigerant circulation device includes
a driving machine that drives the compressor through an accelerator;
an introduction passage that connects the condenser and the driving machine to each other and guides the condensed refrigerant to the driving machine; and
a return passage that connects a primary pipe between the expansion valve and the evaporator to the driving machine and returns the refrigerant having passed through the driving machine to the primary pipe,
wherein the region where the operation temperature of the refrigerant is 150° C. or higher is present in the return passage or around the driving machine.
